(19) United States
(12) Patent Application Publication
(10) Pub. No.: US 2016/0317552 A1
(43) Pub. Date: Nov. 3, 2016

(54) PHARMACEUTICAL FORMULATIONS FOR TREATING MALE SEXUAL DYSFUNCTIONS

(71) Applicant: IMPRIMIS PHARMACEUTICALS, INC., San Diego, CA (US)

(72) Inventors: Mark L. Baum, San Diego, CA (US); Dennis Elias Saadeh, Irvine, CA (US)

(21) Appl. No.: 15/074,259

(22) Filed: Mar. 18, 2016

Related U.S. Application Data
(60) Provisional application No. 62/154,027, filed on Apr. 28, 2015.

(51) Int. Cl. A61K 31/5578; A61K 31/472; A61K 31/522; A61K 31/417

(52) U.S. Cl. CPC A61K 31/5578; A61K 31/417; A61K 31/472; A61K 31/522

(57) ABSTRACT

Pharmaceutical compositions and methods for treating various male sexual dysfunction disorders, such as erectile dysfunction or Peyronie's disease, are described, the compositions comprising a combination of certain pharmaceutically active components (e.g., alprostadil, papaverine, phentolamine and pentoxifylline) and a pharmaceutically acceptable carrier. Methods for fabricating the compositions and using them are also described.

### CROSS REFERENCE TO RELATED APPLICATION(S)

This application claims the benefit of priority under 35 U.S.C. §119(e) of U.S. Ser. No. 62/154,027, filed Apr. 28, 2015, the entire content of which is incorporated herein by reference.

### FIELD OF THE INVENTION

The present invention relates generally to the field of pharmacology and more specifically to compositions and methods designed to treat or mitigate male sexual dysfunctions, and to methods of preparing and using such compositions.

### BACKGROUND

The present disclosure relates to pharmaceutical formulations comprising combinations of active agents such as prostaglandin, vasodilators, alpha-blockers and/or nonspecific phosphodiesterase inhibitor(s), and methods for treating male sexual dysfunctions using such compositions by local administration.

Typical male sexual dysfunctions include erectile dysfunction disorder and Peyronie's disease which are quite widespread. Erectile dysfunction is characterized by the regular or repeated inability to obtain or maintain an erection suitable for sexual intercourse. Many different factors may cause erectile dysfunction including physiological causes (e.g., poor blood flow), side effects of various medications, chronic illnesses such as diabetes, etc. Peyronie's disease is a fibromatosis of the tunica albuginea which usually leads to penile deformation, pain, and often to erectile dysfunction.

Various methods and therapies have been suggested and previously used for the treatment of erectile dysfunction and of Peyronie's disease. In particular, oral administration of nonspecific phosphodiesterase inhibitors has been suggested and tried, but no more than minimal to moderate improvement has been achieved by such methods. Therefore, there remains a need for better treatments of these disorders.

This present disclosure provides such pharmaceutical compositions suitable for treatment and alleviation of erectile dysfunction and of Peyronie's disease that can achieve positive patient outcomes while being free of the drawbacks and deficiencies of existing methods and formulations. Methods of fabricating and administering the same are also provided.

### SUMMARY

According to one embodiment of the invention, there is provided a pharmaceutical composition for treating male sexual dysfunctions, the composition comprising a pharmaceutically active component and a pharmaceutically acceptable carrier, the active component comprising a therapeutically effective quantities of two compounds or pharmaceutically acceptable salts, hydrates, solvates or N-oxides thereof, the first compound being a prostaglandin compound and the second compound being a nonspecific phosphodiesterase inhibitor of formula I:

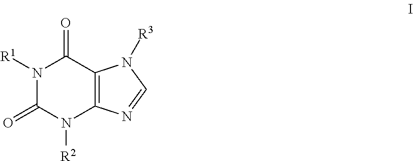

According to another embodiment of the invention, there is provided a further pharmaceutical composition for treating male sexual dysfunctions, wherein the pharmaceutically active component of the composition additionally to the first and second compounds described above further includes a therapeutically effective quantity of an antispasmodic compound having an isoquinoline moiety or pharmaceutically acceptable salts, hydrates, solvates or N-oxides thereof.

According to yet another embodiment of the invention, there is provided a further pharmaceutical composition for treating male sexual dysfunctions, wherein the pharmaceutically active component of the composition additionally to the first, second and antispasmodic compounds described above further includes a therapeutically effective quantity of a non-selective α-adrenergic blocker or pharmaceutically acceptable salts, hydrates, solvates or N-oxides thereof.

According to other embodiments, there are provided specific compounds for making the compositions described above, for example, alprostadil, pentoxifylline, papaverine and phentolamine as well as methods for treating male sexual disorders by local administration of the above-mention composition(s).

### DETAILED DESCRIPTION

### A. Terms and Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees depending on the context. Whenever it appears herein, a numerical range such as “1 to 20” refers to each integer in the given range; i.e., meaning only 1, only 2, only 3, etc., up to and including only 20.

The term “pharmaceutical composition” is defined as a chemical or biological compound or substance, or a mixture or combination of two or more such compounds or substances, intended for use in the medical diagnosis, cure, treatment, or prevention of disease or pathology.

The term male sexual dysfunction is defined herein as any other condition, disease or disorder, regardless of cause or origin, which interferes with male sexual response, such as, but not limited to, erectile dysfunction, Peyronie's disease, priapism and premature ejaculation.

The term “erectile dysfunction” sometimes abbreviated as “ED” refers to one or several conditions associated with deficiencies in male sexual response and is intended to include vasculogenic, neurogenic, endocrinologic or psychogenic impotence in its broadest sense (to indicate a periodic or consistent inability to achieve or sustain an erection of sufficient rigidity for sexual intercourse, particularly vasculogenic impotence).

The term “Peyronie's syndrome” or “Peyronie's disease” is defined as one or several conditions associated with, or caused by, a fibromatosis of the tunica albuginea typically leading to penile deformation (curved penis during erection), pain, and frequent erectile dysfunction.

The terms “solvate” and “hydrate” are used herein to indicate that a compound or substance is physically or chemically associated with a solvent for “solvates” such as water (for “hydrates”).

The term “carrier” refers to a substance that serves as a vehicle for improving the efficiency of delivery and the effectiveness of a pharmaceutical composition.

The term “excipient” refers to a pharmacologically inactive substance that is formulated in combination with the pharmacologically active ingredient of pharmaceutical composition and is inclusive of bulking agents, fillers, diluents and products used for facilitating drug absorption or solubility or for other pharmacokinetic considerations.

The term “therapeutically effective amount” is defined as the amount of the compound or pharmaceutical composition that will elicit the biological or medical response of a tissue, system, animal or human that is being sought by the researcher, medical doctor or other clinician.

The term “pharmaceutically acceptable” is defined as a carrier, whether diluent or excipient, that is compatible with the other ingredients of the formulation and not deleterious to the recipient thereof.

The terms “administration of a composition” or “administering a composition” is defined to include an act of providing a compound of the invention or pharmaceutical composition to the subject in need of treatment.

The terms “local administration” and “locally administering” as used herein refer to treatment of a fibrotic disease by administering at sites proximate to locales where the fibrotic disease manifests itself. It is distinguished from systemic administrations, such as oral administration or intravenous injection, wherein dosage of a pharmaceutical composition is relatively similar throughout the body of a subject. Non-limiting examples of local administration include an injection (e.g., intracavernosal injection), topical administration, and transdermal administration.

### B. Embodiments of the Invention

According to embodiments of the present invention, there are provided pharmaceutical compositions for treating male sexual dysfunctions. The compositions comprise, consist of or consist essentially of a combination of therapeutically effective quantities of at least two of compounds A, B, C and D described below, or combinations of only some of them.

Compound A is a prostaglandin compound or pharmaceutically acceptable salts, hydrates, solvates or N-oxides thereof. Any kind of prostaglandin can be used as compound A, such as, for example, and not limited to, prostaglandin E1, prostaglandin E2, prostaglandin D2, prostaglandin F2, prostaglandin 12, thromboxane or combinations thereof. In one embodiment, the compound A that can be used is alprostadil. Those having ordinary skill in the art can determine if another prostaglandin compound is better suited to the purposes of treating or preventing male sexual disorders and select such alternative compound A, if desired.

Compound B is a nonspecific phosphodiesterase inhibitor of formula I:

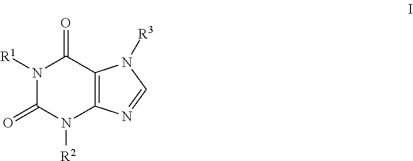

or pharmaceutically acceptable salts, hydrates, solvates or N-oxides thereof. In nonspecific phosphodiesterase inhibitor of formula I, supra, each of R¹, R² and R³ is H, C₁-C₆ alkyl, C₂-C₆ alkenyl, C₂-C₆ alkynyl, cycloalkyl, heterocyclyl, aryl and heteroaryl, each of which is further optionally substituted.

In one non-limiting embodiment the compound B that can be used is pentoxifylline, which is chemically 1-(5-oxohexyl)-3, 7-dimethylxanthine, that is, a compound of formula I where each of R² and R³ is methyl and R¹ is 5-oxohehyl, i.e., a functional group having the structure —(CH₂)₄—4C(O)—CH₃. Lisofylline, an active metabolite of pentoxifylline, i.e., 1-(5-hydroxyhexyl)-3,7-dimethyl-3,7-dimethylxanthine can be also used if desired. The structure of lisofylline is basically the same as that of pentoxifylline except its functional group R¹ includes a primary alcohol moiety —C(OH)— instead of the acyl moiety —C(O)— that is present in the R¹ group in pentoxifylline. Other non-limiting examples of compounds encompassed by formula I that can be used include caffeine, aminophylline, enprofylline, isbufylline, theophylline, theobromine or 3-isobutyl-1-methylxanthine.

The mass ratios between the compound A and the compound B can be between about 1:4,000 and about 1:125, such as between about 1:1,000 and about 1:500, for example, between about 1:800 and about 1:600. The quantity of the compound A in the entire pharmaceutical composition (i.e., the composition that includes not only the active components but also solvents, excipients, adjuvants etc. as discussed below) can be between about 5 micrograms and about 40 micrograms (μg) of the compound A per 1 mL of the entire pharmaceutical composition, such as between about 10 μg/mL and about 30 μg/mL, for example, about 20 μg/mL.

The quantity of the compound B in the entire pharmaceutical composition can be between about 5 mg and about 20 mg of the compound B per 1 mL of the entire pharmaceutical composition. In some embodiments, the therapeutic effective amount of the compound B can be between about 10 mg/mL and about 15 mg/mL, for example, about 12 mg/mL.

Compound C mentioned above is an antispasmodic compound having an isoquinoline moiety or pharmaceutically acceptable salts, hydrates, solvates or N-oxides thereof. In some embodiments, the compound C that can be used in the pharmaceutical compositions mentioned above is papaverine or bis-papaverine.

When a pharmaceutical composition includes compounds A, B and C, the mass ratios between the compound A, the compound B and the compound C can be between about 1:4,000:6,000 and about 1:125:200, such as between about 1:1,000:1,500 and about 1:500:750, for example, between about 1:800:1,200 and about 1:600:900. The quantity of the compound C in the entire pharmaceutical composition can be between about 10 mg and about 30 mg of the compound C per 1 mL of the entire pharmaceutical composition. In some embodiments, the therapeutic effective amount of the compound C can be between about 15 mg/mL and about 20 mg/mL, for example, about 20 mg/mL.

Compound D mentioned above is a non-selective α-adrenergic blocker or pharmaceutically acceptable salts, hydrates, solvates or N-oxides thereof. In some embodiments, compound D that can be used in the pharmaceutical compositions mentioned above is phentolamine, phenoxybenzamine, tolazoline or trazodone.

When a pharmaceutical composition includes compounds A, B, C and D, the mass ratios between the compound A, the compound B, the compound C and the compound D can be between about 1:4,000:6,000:400 and about 1:125:200:15, such as between about 1:1,000:1,500:100 and about 1:500:750:50, for example, between about 1:800:1,200:80 and about 1:600:900:60. The quantity of the compound D in the entire pharmaceutical composition can be between about 0.5 mg and about 2.0 mg of the compound D per 1 mL of the entire pharmaceutical composition. In some embodiments, the therapeutic effective amount of the compound D can be between about 1.5 mg/mL and about 2.0 mg/mL, for example, about 2.0 mg/mL.

In certain embodiments, the pharmaceutical formulation may further optionally comprise one or more additional active agent(s) other than those mentioned above. In particular embodiments, the second active agent can be (an)other vasodilator(s), e.g., α-receptor blocking agent(s), ergot alkaloid(s), antihypertensive agent(s), naturally occurring, semisynthetic and synthetic prostaglandin(s) and/or vasoactive intestinal peptide(s). In other embodiments, the pharmaceutical formulation may further comprises a collagenase, such as collagenase clostridium histolyticum.

As mentioned above, the pharmaceutical compositions described herein may contain not only pharmaceutically active components (that is, a combination of compounds A, B C and/or D or of some of them, plus optional additional active compounds, if any) but also, in some embodiments, may further comprise one or several pharmaceutically acceptable excipient(s) or carrier(s), including, but not limited to, antioxidant(s), adjuvant(s), synergist(s) and/or preservative(s).

Non-limiting examples of the antioxidant that can be used include α-tocopherol acetate, acetone sodium bisulfite, acetylcysteine, ascorbic acid, ascorbyl palmitate, butylated hydroxyanisole, butylated hydroxytoluene, cysteine, cysteine hydrochloride, d-α-tocopherol natural, d-α-tocopherol synthetic, dithiothreitol, monothioglycerol, nordihydroguaiaretic acid, propyl gallate, sodium bisulfite, sodium formaldehyde sulfoxylate, sodium metabisulfite, sodium sulfite, sodium thiosulfate, thiourea, tocopherols.

Non-limiting examples of the adjuvant or synergist include citric acid, EDTA (ethylenediaminetetraacetate) and salts, hydroxyquinoline sulfate, phosphoric acid, and tartaric acid.

In those embodiments where the formulation includes an EDTA sodium salt as an adjuvant, the EDTA sodium salt can be 0-0.15% by weight of the formulation, for example, 0.01, 0.02, 0.03, 0.04, 0.05, 0.06, 0.07, 0.08, 0.09, 0.10, 0.11, 0.12, 0.13, 0.14, or 0.15% by weight of the formulation. If an EDTA magnesium salt is used salt as an adjuvant, the EDTA magnesium salt can be 0-0.15% by weight of the formulation, for example, 0.01, 0.02, 0.03, 0.04, 0.05, 0.06, 0.07, 0.08, 0.09, 0.10, 0.11, 0.12, 0.13, 0.14, or 0.15% by weight of the formulation.

Non-limiting examples of the preservative are benzalkonium chloride, benzethonium chloride, benzoic acid and salts, benzyl alcohol, boric Acid and salts, cetylpyridinium chloride, cetyltrimethyl ammonium bromide, chlorobutanol, chlorocresol, chorhexidine gluconate or chlorhexidine acetate, cresol, ethanol, imidazolidinyl urea, metacresol, methylparaben, nitromersol, o-phenyl phenol, parabens, phenol, phenylmercuric acetate/nitrate, propylparaben, sodium benzoate, sorbic acids and salts, β-phenylethyl alcohol, thimerosal. In particular embodiments, the preservative is benzyl alcohol.

In those embodiments where the formulation includes ethanol as a preservative, ethanol can be 190 proof. The ethanol can be 0-15% by volume of the formulation, for example, 0, 1, 2, 3, 4, 5, 6, 7, 8, 9, 10, 11, 12, 13, 14, or 15% by volume of the formulation. In those embodiments where the formulation includes benzyl alcohol as a preservative, benzyl alcohol can be 0-1.5% by weight of the formulation, for example, 0, 0.1, 0.2, 0.3, 0.4, 0.5, 0.6, 0.7, 0.8, 0.9, 1.0, 1.1, 1.2, 1.3, 1.4, or 1.5% by volume of the formulation.

In some embodiments, the pharmaceutical formulation is filtered before local administration. In particular embodiments, the pharmaceutical formulation is filtered through a 0.22 micron filter before local administration. In other embodiments, the pharmaceutical formulation has a pH of between 4 and 8. In particular embodiments, the pharmaceutical formulation has a pH of between 5.5 and 6. The pH can be adjusted by adding acids or bases, e.g., HCl or NaOH.

The pharmaceutical formulations that are described herein may in addition optionally contain other pharmacologically active compounds such as at least one anti-bacterial agent(s), at least one antiviral medicament(s) and combinations thereof. Those having ordinary skill in the art can determine what specific anti-bacterial and/or antiviral medicament(s) are to be used, if any.

The concentration of the anti-bacterial agent(s) in the compositions of the present application may be between about 0.01 mg/mL and about 50.0 mg/mL, such as between about 0.5 mg/mL and about 10.0 mg/mL, for example, about 1.0 mg/mL. Non-limiting examples of the anti-bacterial agents that may be used include fluoroquinolones such as moxifloxacin, gatifloxacin, nalidixic acid, oxolinic acid, piromidic acid, pipemidic acid, rosoxacin, enoxacin, fleroxacin, lomefloxacin, nadifloxacin, ofloxacin, pefloxacin, rufloxacin, balofloxacin, levofloxacin, norfloxacin, ciprofloxacin, pazufloxacin, sparfloxacin, tosufloxacin, clinafloxacin, gemifloxacin, sitafloxacin, prulifloxacin and combinations thereof.

Non-limiting examples of anti-bacterial agents other than fluoroquinolones that may be used include vancomycin, teicoplanin, telavancin, decaplanin, ramoplanin, azitromycin, gentamicin, tobramycin, amikacin, cefuroxime, mitomycin, neomycin, neosporin, amoebicides (e.g., metronidazole, tinidazole, secnidazole, orornidazole, polyhexamethylene biguanide or chlorohexidine), polymyxin, clindamycin, bacitracin, chloramphenicol, erythromycin, natamycin, blephamide, sulfacetamide, sodium bicarbonate, povidone-iodine and combinations thereof.

The concentration of the antiviral medicament(s) in the compositions of the present application may be between about 0.01 mg/mL and about 75.0 mg/mL, such as between about lmg/mL and about 50.0 mg/mL, for example, about 20.0 mg/mL. Non-limiting examples of the antiviral medicaments that may be used include idoxuridine, vidarabine and combinations thereof.

As mentioned above, the pharmaceutical composition that is the subject matter of the instant application may further optionally include one or several pharmaceutically acceptable excipient(s). In some embodiments, an excipient that can be used may be a non-ionic polyoxyethylene-polyoxypropylene block copolymer having the following general structure:

HO—(CH₂—CH₂—O)ₓ—(C₃H₆—O)_{y}—(CH₂—CH₂—O)ₓ—H,

wherein x is an integer having the value of at least 8 and y is an integer having the value of at least 38.

If a non-ionic polyoxyethylene-polyoxypropylene block copolymer is used as an excipient, its contents in the overall composition may be between about 0.01 mass % and about 20.0 mass % such as between about 1.0 mass % and about 15 mass %, for example, about 10.0 mass %.

One non-limiting example of a specific non-ionic polyoxyethylene-polyoxypropylene block copolymer that can be used as a solubilizing and stabilizing agent in the pharmaceutical compositions of the instant invention is the product known under the trade name Poloxamer 407® (poly(ethylene glycol)-block-poly(propylene glycol)-block-poly(ethylene glycol)) available from Sigma-Aldrich Corp. of St. Louis, Mo., with the molecular weight of the polyoxypropylene portion of about 4,000 Daltons, about a 70% polyoxyethylene content, the overall molecular weight of between about 9,840 Daltons and about 14,600 Daltons and having the following chemical structure:

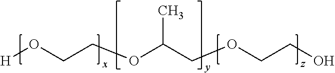

According to further embodiments, the excipient portion of the pharmaceutical formulation may contain other products, instead of, or in combination with, non-ionic polyoxyethylene-polyoxypropylene block copolymer(s). One non-limiting example of such additional excipient is poly(acrylic acid) in its various cross-linked or non-cross-linked versions, such as Carbomer 940® having a weight-average molecular weight of about 940 and available from Lubrizol Corp. of Wickliffe, Ohio. Another type of products that can be used in the excipient portion of the pharmaceutical formulation may be water-soluble methylcellulose and hydroxypropyl methylcellulose polymers, such as Methocel® family of products available from Dow Chemical Co. of Midland, Mich., for example, a hydroxypropyl methylcellulose product Methocel® E4M.

The pharmaceutical formulation can be administered to a subject in need thereof by various local administrations for treating and or preventing male sexual disorders described above. In one non-limiting embodiment, the local administration is by intracavernosal injection one to several times in a twenty-four hour period, the precise regimen to be determined by a reasonably skilled practitioner. In other embodiments, the pharmaceutical formulation is administered topically, e.g., transdermally, to be applied as a cream, a gel or an ointment.

It will be understood by those having ordinary skill in the art that the specific dose level and frequency of dosage for any particular patient may be varied and will depend upon a variety of factors including the activity of the specific compound employed, the metabolic stability and length of action of that compound, the age, body weight, general health, gender, diet, and the severity of the particular ophthalmological condition being treated.

The methods provided herein can be used as a mono therapy or a part of a combo therapy. In certain embodiments, the pharmaceutical formulations described above can be used as a mono therapy. In other embodiments, such formulations can be used as a part of a combo therapy, for example, when the formulations are used, in combination with a collagenase therapy, e.g., collagenase clostridium histolyticum or Xiaflex®.

According to further embodiments, methods for fabricating the above-described pharmaceutical compositions are provided. A one-batch formulation method may be used, where the components of the pharmaceutical formulation can be combined in single container; the components may be added to the container simultaneously or consecutively. Alternatively, a two- or multiple-batch method(s) may be used if desired, where each component of the pharmaceutical formulation can be combined in separate container followed by combining the contents of each container.

In one exemplary, non-limiting procedure, pre-measured quantities of alprostadil, papaverine hydrochloride, phentolamine mesylate, pentoxifylline and sodium metabisulfite (as an antioxidant serving as a preservative) may be mixed with water to form a solution suitable for injections. The solution can be then used for treating various male sexual dysfunctions described above. Alternatively, the composition can be formulated as a gel, a cream or an ointment that can be used for treating the same by topical (urethral) instillation. To fabricate such topical preparation, the solution for injection described above can be further mixed with a vehicle to be selected by those having ordinary skill in the art. Typically, a 2-3% aqueous solution of hydroxyethylcellulose may be used as the vehicle.

In further embodiments, the pharmaceutical formulations discussed above may be formulated in the lyophilized form, e.g., to insure a longer shelf life. It is within the knowledge of those having ordinary skill in the art to determine the precise procedure to be used for lyophilization. In one exemplary, non-limiting procedure, an above-described aqueous solution may be optionally de-gassed followed by freezing at low temperatures (e.g., −70° C. or lower) for a period of time (e.g., at least 24 hours) to allow the solution to completely solidify and to form proper ice crystals. Then, the frozen composition is subjected to a vacuum of at least 30 mm Hg for a period of time (e.g., at least 24 hours). As a result of exposure to the vacuum, the water sublimates from the frozen solution (i.e., transitions from the solid to the gas phase bypassing the liquid phase) to form a completely dry, powder-like substance which is the lyophilized composition.

The lyophilized dry composition prepared as described above is expected to remain stable for at least several months and for as long as two years. Stability can be evaluated by those having ordinary skill in the art according to one or more of various methods, e.g., by the retention of potency after storage for a period of time, by showing that no loss (or not more than negligible loss) of concentration of epinephrine has occurred during the period of storage, or by visual observations such as no change in color.

Next, the lyophilized dry composition prepared as described above is to be reconstituted immediately prior to its use, for example, 1 hr or less before being administered, or 30 minutes or less. To reconstitute the composition, a quantity of sterile de-ionized water for injection is to be added to the dry composition and a clear solution can thus obtained. The concentration of the active components (i.e., a combination of the compounds A, B and/or C and/or D plus optional additional active compounds, if any) that is desirable to have for the injection determines the quantity of water to be added to the dry composition, to be chosen by one having ordinary skill in the art.

In other optional embodiments that can be also practiced, the pharmaceutical composition may comprise a fully lyophilized portion, a non-lyophilized portion and, optionally, an extra quantity of pure sterile de-ionized water. Those having ordinary skill in the art can determine the quantities of the fully lyophilized and non-lyophilized portions and of water so as to achieve the desired ratios of the ingredients and their concentrations.

A variety of apparatuses can be used for reconstituting the composition. For example, water can be added to the dry composition in a single container such as a vial. Once a clear solution has been formed, it can be collected by a syringe. Alternatively, the dry composition can be mixed with sterile water or with a balanced salt solution in an infusion bottle, to be delivered in the phacoemulsification procedure if desired.

In additional embodiments, pharmaceutical kits are provided. The kit includes a sealed container approved for the storage of pharmaceutical compositions, the container containing one of the above-described pharmaceutical compositions and a device for locally administering the formulation (e.g., a syringe and a needle). An instruction for the use of the composition and the information about the composition are to be affixed to the container or otherwise enclosed with it.

The following examples are provided to further elucidate the advantages and features of the present invention, but are not intended to limit the scope of the invention. The examples are for the illustrative purposes only. USP pharmaceutical grade products were used in preparing the formulations described below.

### EXAMPLE 1

### Preparing a Pharmaceutical Composition

A pharmaceutical composition may be prepared as described below. The following products can be used in the amounts and concentrations specified:

(a) about 0.025 mL of an aqueous solution of alprostadil (i.e., prostaglandin El), at a concentration of about 2.0 mg/mL;

(b) about 5.0 mg of phentholamine mesylate;

(c) about 100.0 mg of papaverine hydrochloride;

(d) about 5.0 mL of an aqueous solution of pentoxifylline, at a concentration of about 10.0 mg/mL; and

(e) about 5.0 mL of sterile water for injection.

These components can be combined and allowed to spin for about 30 minutes or until clear solution is obtained. The solution can be filtered through a 0.22 micron filtered transferred into de-pyrogenated, pre-sterilized single dose vials (2 mL size), capped and sealed. Complete sterility and endotoxin testing can be performed by an outside laboratory to ensure safety.

### EXAMPLE 2

### Preparing a Lyophilized Pharmaceutical Composition

A pharmaceutical composition may be prepared as described below. The following products can be used in the amounts and concentrations specified. First, a solution comprising alprostadil, phentholamine, papaverine and pentoxifylline can be prepared as described in Example 1. To this solution about 50.0 mg of powdered mannitol can be added.

The resulting composition can be placed into a commercial lyophilizer where it can be freeze-dried using any standard lyophilization protocol to be selected by those having ordinary skill in the art. For example, the solution obtained as described above can be filtered through 0.22 micron Supor filters into depyrogenated, sterile vials, partially capped with three prong lyophilization stoppers and immediately frozen at about −71° C. for about 24 hours to allow complete freezing and proper ice crystal formation. Degassing of solution prior to freeze-drying is merely optional.

Frozen vials can then be placed into a vacuum chamber, vacuum be drawn and freeze drying process may be commenced. Two stage drying process (primary and secondary drying) over the next 24 hours may take place during this time. When completed, vials may be fully stoppered and sealed followed by optional chromatographic testing (HPLC) for potency and stability after being stored for some time. The lyophilized composition can then be re-constituted about 30 minutes prior to being administered to a patient by adding a quantity of sterile water for injection to achieve the desired concentration of active ingredients in the solution.

Although the invention has been described with reference to the above examples, it will be understood that modifications and variations are encompassed within the spirit and scope of the invention. Accordingly, the invention is limited only by the following claims.

What is claimed is:

1. A pharmaceutical composition for treating male sexual dysfunctions, comprising a pharmaceutically active component comprising a combination of compounds or pharmaceutically acceptable salts, hydrates, solvates or N-oxides thereof, the combination selected from the group consisting of:
(a) a combination of compound A and compound B;
(b) a combination of compound A and compound C;
(c) a combination of compound B and compound D;
(d) a combination of compound C and compound D;
(e) a combination of compound A, compound B and compound C; and
(f) a combination of compound B, compound C and compound D; and
(g) a combination of compound A, compound B, compound C and compound D,
wherein compound A is a prostaglandin compound;
compound B is a nonspecific phosphodiesterase inhibitor of formula I: 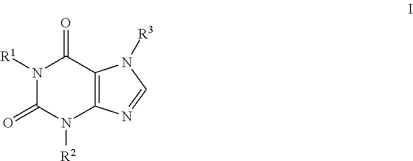
wherein each of R¹, R² and R³ is independently selected from the group consisting of H, C₁-C₆ alkyl, C₂-C₆ alkenyl, C₂-C₆ alkynyl, cycloalkyl, heterocyclyl, aryl and heteroaryl, each of which is further optionally substituted;
compound C is an antispasmodic compound having an isoquinoline moiety; and
compound D is a non-selective α-adrenergic blocker.

2. The pharmaceutical composition of claim 1, wherein the prostaglandin compound is selected from the group consisting of prostaglandin E1, prostaglandin E2, prostaglandin D2, prostaglandin F2, prostaglandin 12 and thromboxane.

3. The pharmaceutical composition of claim 2, wherein the prostaglandin compound is alprostadil.

4. The pharmaceutical composition of claim 1, wherein each of R¹, R² and R³ is independently selected from the group consisting of H, a C₁-C₆ alkyl and an acyl-substituted C₁-C₆ alkyl.

5. The pharmaceutical composition of claim 1, wherein the nonspecific phosphodiesterase inhibitor is selected from the group consisting of pentoxifylline, caffeine, aminophylline, enprofylline, isbufylline, theophylline, theobromine and 3-isobutyl-1-methylxanthine.

6. The pharmaceutical composition of claim 5, wherein the nonspecific phosphodiesterase inhibitor is pentoxifylline. The pharmaceutical composition of claim 1, wherein the antispasmodic compound is selected from the group consisting of papaverine and bis-papaverine.

8. The pharmaceutical composition of claim 7, wherein the antispasmodic compound is papaverine.

9. The pharmaceutical composition of claim 1, wherein the non-selective α-adrenergic blocker is selected from the group consisting of phentolamine, phenoxybenzamine, tolazoline and trazodone.

10. The pharmaceutical composition of claim 9, wherein the non-selective α-adrenergic blocker is phentolamine.

11. The pharmaceutical composition of claim 1, wherein the composition is formulated as a solution suitable for injections.

12. The pharmaceutical composition of claim 1, wherein the composition is formulated as a cream, an ointment or a gel.

13. The pharmaceutical composition of claim 1, wherein the composition is lyophilized.

14. The pharmaceutical composition of claim 13, wherein the composition is re-constituted by adding a quantity of sterile water for injection prior to the composition being administered.

15. A method for treating a male sexual dysfunction, comprising locally administering to the subject the pharmaceutical composition of claim 1.

16. The method of claim 15, wherein the dysfunction is selected from the group consisting of erectile dysfunction disorder, Peyronie's disease, priapism and premature ejaculation.

17. The method of claim 15, wherein the local administering is selected from the group consisting of intercavernosal injection and topical application.

18. A kit comprising:
(a) the pharmaceutical composition of claim 1;
(b) a device for locally administering the composition;
(c) a container for housing the composition and the delivery device; and
(d) a label and instructions for use affixed to, or enclosed with, the container.

19. A pharmaceutical formulation comprising the pharmaceutical composition of claim 1 and a pharmaceutically acceptable carrier.
